(19) United States
(12) Patent Application Publication
(10) Pub. No.: US 2009/0035351 A1
(43) Pub. Date: Feb. 5, 2009

(54) Bioabsorbable Hypotubes for Intravascular Drug Delivery

(75) Inventors: Joseph Berglund, Santa Rosa, CA (US); Ankit Shah, Emeryville, CA (US); Feridun Ozdil, Santa Rosa, CA (US); Christopher Bonny, Eugene, OR (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/212,817

(22) Filed: Sep. 18, 2008

Related U.S. Application Data
(63) Continuation-in-part of application No. 11/780,702, filed on Jul. 20, 2007.

(51) Int. Cl. A61F 2/82

(52) U.S. Cl. 424/426; 623/1.42

(57) ABSTRACT

A biodegradable implantable device for delivering a drug to a treatment site includes a biodegradable hypotube defining a lumen and at least one drug disposed within the lumen of the hypotube. At least one drug is released from the lumen upon degradation of the biodegradable hypotube. The lumen may be compartmentalized, each compartment containing a different drug. The hypotube may also include a plurality of pores in fluid communication with the compartments providing different drug release profiles.

### RELATED APPLICATIONS

This application is a continuation-in-part application claiming priority to, and the benefit of, U.S. patent application Ser. No. 11/780,702 titled Hypotubes for Intravascular Drug Delivery, to Feridun Ozdil, et al., filed Jul. 20, 2007, the entirety of which is incorporated herein by reference.

### FIELD OF THE INVENTION

The present invention relates to drug-eluting implantable devices for intravascular drug delivery.

### BACKGROUND OF THE INVENTION

Stenosis is the narrowing of an anatomical passageway or opening in the body, such as seen in blood vessels. A number of physiological complications have been associated with stenosis, such as ischemia, cardiomyopathy, angina pectoris, and myocardial infarction. In response, several procedures have been developed for treating stenosis. For example, in percutaneous transluminal coronary angioplasty (PTCA), a balloon catheter is inserted into a blocked or narrowed coronary blood vessel of a patient. Once the balloon is positioned at the blockage or narrowing, the balloon is inflated causing dilation of the vessel. The catheter is then removed from the site to allow blood to more freely flow through the less restricted vessel.

While the PTCA procedure has proven successful in treating stenosis in the past, several shortcomings associated with the procedure have been identified. For example, an ongoing problem with PTCA is that in about one-third of cases, the blockage or narrowing of the vessel returns often within about six months of initial treatment. It is thought that the mechanism of this “relapse,” called “restenosis,” is not solely the progression of coronary artery disease, but rather the body's immune system response to the “injury” caused by the procedure. For instance, PTCA often triggers blood clotting (i.e., “thrombosis”) at the site of the procedure resulting in re-narrowing of the vessel. In addition, tissue growth at the site of treatment caused by an immune system response in the area also can occur and result in re-narrowing of the vessel. This tissue growth—a migration and proliferation of the smooth muscle cells that are normally found in the media portion of the blood vessel (i.e., neointimal hyperplasia)—tends to occur during the first three to six months after the PTCA procedure, and it is often thought of as resulting from “over exuberant” tissue healing and cellular regeneration after the PTCA procedure.

Stents and/or drug therapies, either alone or in combination with the PTCA procedure, are often used to avoid or mitigate the effects or occurrence of restenosis. In general, stents are mechanical scaffoldings which may be inserted into a blocked or narrowed region of a passageway to provide and maintain its patency. During implantation, a stent can be positioned on a delivery device (for example and without limitation a balloon catheter) and advanced from an external location to an area of passageway blockage or narrowing within the body of the patient. Once positioned, the delivery device can be actuated to deploy the radially expandable stent. Expansion of the stent can result in the application of force against the internal wall of the passageway, thereby improving the patency of the passageway. Thereafter, the delivery device can be removed from the patient's body.

Stents may be manufactured in a variety of lengths and diameters and from a variety of materials ranging from metallic materials to polymers. Stents may also incorporate and release drugs (i.e., “drug-eluting stents”) that can affect endothelialization as well as the formation of and treatment of existing plaque and/or blood clots. In some instances then, drug-eluting stents can reduce, or in some cases, eliminate, thrombosis and/or restenosis. In still other instances, drug-eluting stents can promote or encourage endothelialization.

Drug-eluting stents generally carry and release drugs in polymer matrices applied to the surfaces of the stent during or after its manufacture thereby forming one or more layers of stent coatings that elute the carried drug(s) once implanted at a treatment site. Thus, positioning the drug-eluting stent at a target site enables localized delivery of the drugs to the target site while providing radial support to its structure.

Although drug-eluting polymer stent coatings can be beneficial for the treatment of stenosis or restenosis, they suffer from several limitations. For example, the maximum polymer coating thickness is generally limited to about 10 to 50 microns. Therefore, the effective amount and duration of drug release is limited to the amount of drug(s) that can be included within the particular thickness of a coating.

Another limitation for stent coatings is that drug coatings applied to a stent surface are fragile and may be damaged or otherwise compromised during manufacture, packaging and delivery to the treatment site. Damage to the drug coating may result in a loss of a portion of the drug thereby reducing the effective amount of drug available for release after implantation.

In light of the foregoing, there is an ongoing need for biodegradable implantable devices such as stents that are capable of both providing sufficient radially expanding force to a passageway while delivering drugs. The present invention addresses these needs, among others.

### BRIEF SUMMARY OF THE INVENTION

One aspect of the present invention provides a biodegradable implantable device for delivering a drug to a treatment site. The implantable device includes a biodegradable hypotube defining a lumen and at least one drug disposed within the lumen of the hypotube. At least one drug is released from the lumen of the biodegradable hypotube. In one embodiment, at least one drug is released from the lumen upon degradation of the biodegradable hypotube. The lumen may be compartmentalized, each compartment containing a different drug. The hypotube may also include a plurality of pores in fluid communication with the compartments providing different drug release profiles.

The foregoing and other features and advantages of the invention will become further apparent from the following detailed description of the presently preferred embodiments, read in conjunction with the accompanying drawings. The drawings are not necessarily drawn to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 illustrates perspective and partial longitudinal cross-section views of one embodiment of an implantable device made in accordance with the present invention.

FIGS. 2a and 2b illustrate cross-section views of an exemplary stent from two perspectives, crosswise (FIG. 2a) and lengthwise (FIG. 2b), of another embodiment of an implantable device made in accordance with the present invention.

FIG. 3 illustrates another embodiment of an implantable device made in accordance with the present invention.

FIG. 4 illustrates another embodiment of an implantable device made in accordance with the present invention.

FIG. 5 illustrates another embodiment of an implantable device made in accordance with the present invention.

FIG. 6 illustrates another embodiment of an implantable device made in accordance with the present invention.

FIG. 7 illustrates another embodiment of an implantable device made in accordance with the present invention.

### DETAILED DESCRIPTION OF THE INVENTION

The present invention provides biodegradable drug-eluting implantable devices for intravascular drug delivery. The present invention provides this advance by providing implantable devices, including stents, that comprise one or more tubes (referred to herein as “hypotubes”) within or around the structure of the device. These hypotubes contain one or more drugs that can elute through either the walls of the tubes (i.e., diffusive transport) and/or one or more openings or pores (hereinafter “pores”) disposed within a wall of the hypotube. In other embodiments described below, a drug contained within a lumen of a biodegradable hypotube is released when the hypotube degrades. In still other embodiments, a drug contained within a lumen of a biodegradable hypotube is released prior to the degradation of the hypotube.

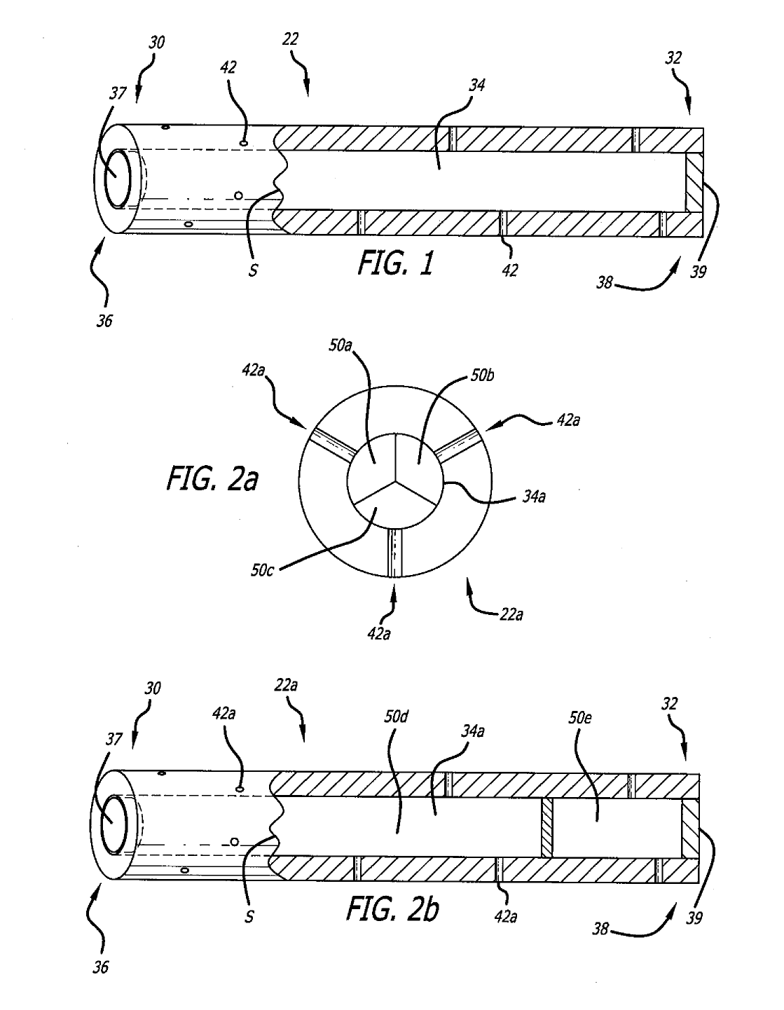

FIG. 1 illustrates a partial longitudinal cross section of one embodiment of a hypotube made in accordance with the present invention. As shown in FIG. 1, hypotube 22 has a proximal end 30 and a distal end 32. As shown in the cross-section view of FIG. 1 (to the right of line S), hypotube 22 also has a lumen 34 extending between proximal end 30 and distal end 32. In one embodiment, hypotube 22 also comprises proximal opening 36 and distal opening 38, each of which can be in fluid communication with lumen 34. In one embodiment, one or more pores 42 formed on hypotube 22 are in fluid communication with lumen 34, as shown by the cross-section view of FIG. 1. Pores 42 are formed by any method such as, for example, by using an excimer laser to achieve the preferred diameter and depth. Pores 42 can comprise any appropriate shape, such as, for example, circular, elliptical or rectangular configurations.

In one embodiment, hypotube 22 is formed from a metal, a metal alloy, a polymer or a combination thereof. In another embodiment, the hypotube is formed from a non-erodable polymeric material selected from the group consisting of polyether sulfone; polyamide; polycarbonate; polypropylene; high molecular weight polyethylene; polydimethylsiolxane, poly(ethylene-vinylacetate); acrylate based polymers or copolymers, e.g., poly(hydroxyethyl methylmethacrylate; polyvinyl pyrrolidinone; fluorinated polymers such as polytetrafluoroethylene; cellulose esters; and the like. Furthermore, the hypotube may also be formed of a semi-permeable or microporous material. In non-erodible hypotubes, the materials for covering or plugging hypotube pores can be biodegradable or non-erodible materials as disclosed herein.

As shown in FIG. 1, distal opening 38 can be covered or plugged, for example, using weld 39, or another appropriate means for covering or plugging the opening. One or more drugs can be loaded into lumen 34 through proximal opening 36, for example, using a syringe or any other suitable means. In another embodiment, proximal opening 36 can be covered or plugged, for example, using weld 37, or another appropriate means for covering or plugging the opening. One or more drugs can also be loaded into hypotube 22 through one or more pores 42 as appropriate or by other means which will be apparent to one of ordinary skill in the art. Distal opening 38 and proximal opening 36 can be covered or plugged with a biodegradable or biostable material.

As used herein, “drug” shall include any compound or bioactive agent having a therapeutic effect in an animal. The one or more drug loaded into the hypotube may be selected from the group consisting of anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like. Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid and zotarolimus (ABT-578). Additionally, other rapamycin hydroxyesters may be used in the present invention.

In one embodiment, one or more drugs elute through one or more pores 42. In another embodiment, one or more pores 42, the distal opening 38, and/or the proximal opening 36, can initially be covered or plugged with a biocompatible material that can biodegrade or bioerode over time allowing freer drug elution over time. To further affect drug release, varying thicknesses of the biocompatible biodegradable or bioerodable material can be used to cover or plug the one or more pores 42, the distal opening 38, and/or the proximal opening 36.

In one embodiment, hypotube 22 is coated with one or more layers of biocompatible material to cover or plug the one or more pores 42, the distal opening 38, and/or the proximal opening 36, and the one or more layers of biocompatible biodegradable material can biodegrade, bioerode, and/or otherwise dissociate from hypotube 22 to allow for drug release through the one or more pores 42, the distal opening 38, and/or the proximal opening 36 of hypotube 22.

The biodegradable material used to cover or plug the one or more pores 42, distal opening 38, and/or the proximal opening 36 is a material selected from the group consisting of biodegradable metals, metal alloys and polymers. In one embodiment, the biodegradable polymer is selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, and combinations thereof.

In one embodiment, the biodegradable material used to cover or plug the one or more pores 42, distal opening 38, and/or the proximal opening 36 includes a therapeutic agent. In one embodiment, the drug included in the plug material has a drug release profile that provides an initial burst of drug upon implantation of the medical device.

In one embodiment, distal opening 38 and proximal opening 36 are covered or plugged with a biostable material and one or more pores 42 are covered or plugged with a biodegradable material. In another embodiment, the pores are plugged with a biodegradable polymer such as, for example, poly-lactide-co-glycolide or poly-L-lactide-co-caprolactone.

In one embodiment, one or more drugs can be combined with a carrier, such as a biocompatible polymer to alter the release profile of the drug. The carrier can biodegrade or bioerode over a period of time to allow drug-elution to occur more freely over time. In another specific, non-limiting example, the carrier is generally nonbiodegradable, or biostable, that can allow drug to separate from the carrier over time (e.g., via diffusion) for controlled drug delivery.

In one embodiment, the biocompatible carrier comprises a biodegradable material selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, and combinations thereof. In another embodiment, the biocompatible carrier comprises a liquid-based carrier such as, for example, mineral oils, caster oils, and ethylene glycol. In another embodiment, the biocompatible carrier includes a stabilizer such as BHT.

It is contemplated that drug and/or drug/carrier can be in a variety of physical forms, including and without limitation, liquid, solid, gel and combinations thereof, when they are loaded into lumen 34 of hypotube 22. Accordingly, in some embodiments (e.g., when drug and/or drug/carrier are in a liquid form), it may be necessary to cover or plug one or more pores 42, the distal opening 38, and/or the proximal opening 36, before and/or after the drug and/or drug/carrier are loaded into lumen 34 to retain the drug and/or drug/carrier within lumen 34 for a specific amount of time (e.g., until after its deployment to a treatment site).

Further, in accordance with the present invention, any number of drug and/or drug/carrier combinations are envisioned and it is not intended that merely one or two different drugs and/or drug/carrier be employed.

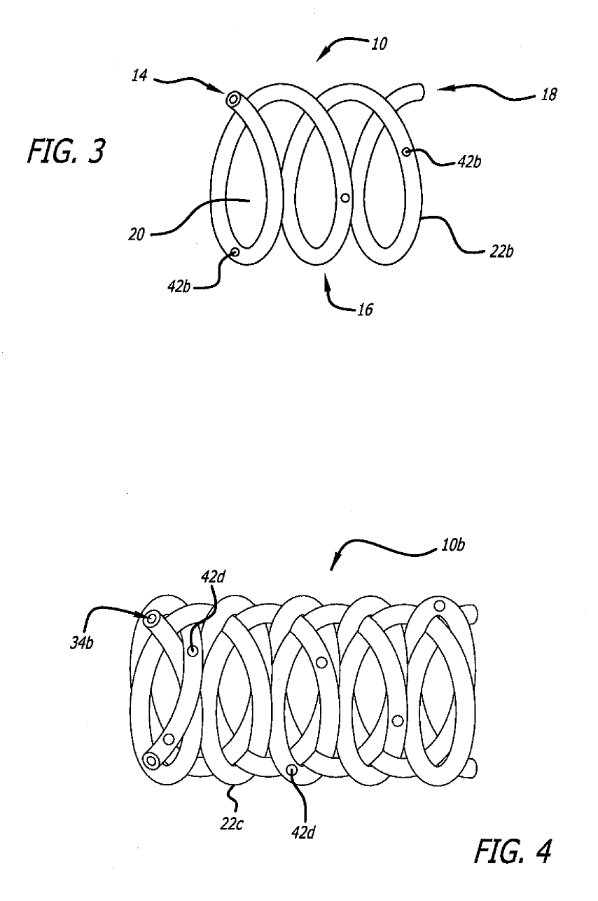

In keeping with this aspect of the present invention, note that in certain embodiments, as shown in FIG. 2a, hypotube lumen 34a can be compartmentalized into one or more discrete spaces, for example, compartments 50a, 50b and 50c, to provide areas of the hypotube for different uses. These compartmentalized spaces can be used to more precisely control areas of drug release or can be used to house and release different drugs that cannot co-exist within the same space due to various incompatibilities. Likewise, and as described previously, different compartmentalized areas of a particular hypotube can exhibit similar or different drug release profiles. While FIG. 2a depicts hypotube 22a having three compartments, the present invention includes embodiments of hypotube 22a having more or less compartments. In one embodiment, hypotube 22a contains two compartments. In another embodiment, hypotube 22a contains four compartments. In another embodiment, depicted in FIG. 2b, the hypotube is compartmentalized along its long axis rather than along its azimuthal coordinates into two or more compartments, in a non-limiting example compartments 50d and 50e.

FIG. 3 illustrates one embodiment of an implantable device 10 made in accordance with the present invention. For convenience and brevity, the device depicted in FIG. 3 is a stent. However, it should be noted that other devices or prostheses are also within the scope of the claimed invention. As shown in FIG. 3, stent 10 includes one or more hypotubes 22b that form the body of stent 10. Those skilled in the art will appreciate that hypotubes 22b can be manipulated to form a variety of suitable patterns in forming stent 10, including without limitation, in straight, sinusoidal, coiled, helical, zig-zag, filament type, or V-shaped patterns. Furthermore, a plurality of hypotubes 22b can be formed into stent 10 such that the plurality of hypotubes 22b forms a multiple helix, a braid, a mesh or a woven configuration. As also shown in FIG. 3, stent 10 can be cylindrical or tubular in shape and can have a first end 14, a midsection 16, and a second end 18. Additionally, a hollow channel 20 extends longitudinally through the body structure of the stent 10. The structure of stent 10 allows insertion of stent 10 into a body passageway where stent 10 can physically hold open the passageway by exerting a radially outward-extending force against the walls or inner surface of the passageway. If desired, stent 10 can also expand the opening of the passageway to a diameter greater than the passageway's original diameter and, thereby, increase fluid flow through the passageway. As shown in FIG. 3, hypotube 22b can comprise one or more pores 42b to release drugs contained therein. Alternatively, or in combination, drugs can be released from ends 14 and/or 18, when, for example, one or both of these ends are not covered or plugged as described above.

Drug release profiles and the particular location of drug release can also be controlled by varying the number, size, and/or placement of pores on a particular hypotube. In one embodiment, to reduce or eliminate the incidence of smooth muscle cell proliferation and/or restenosis, the number and/or size of pores can be increased along the channel of the stent for eluting drugs that reduce or prevent cell migration to the channel of the stent. The number and/or size of pores can also be increased at the sites proximal to the walls or inner surface of the passageway for eluting drugs that promote healing of the walls and/or reduce platelet sequestration due to implantation-related injuries.

As previously indicated, those skilled in the art will appreciate that an implantable device according to the present invention (such as a stent) may be manufactured in a variety of sizes, lengths, and diameters (inside diameters as well as outside diameters). A specific choice of size, length, and diameters depends on the anatomy and size of the target passageway, and can vary according to intended procedure and usage. In another embodiment, the implantable device is in a configuration selected from the group consisting of a helical configuration, a braided configuration, a mesh configuration and a woven configuration. In another embodiment, the implantable device comprises more than one hypotube. In another embodiment, the implantable device comprises two or more hypotubes in a configuration selected from the group consisting of a helical configuration, a braided configuration, a mesh configuration and a woven configuration. Those skilled in the art will also appreciate that the hypotube and or the lumen inside the hypotube may have a cross section other than the circular cross section illustrated. For example, a hypotube and or the lumen may have a square, rectangular or oval cross section. In other embodiments, the cross section of the hypotube may be different than the cross section of the lumen. For example, the hypotube may have a generally rectangular cross section and the lumen with the hypotube may have a generally oval cross section. Those with ordinary skill in the art will appreciate that there are many combinations of various shapes of the hypotube and the lumen running through the hypotube.

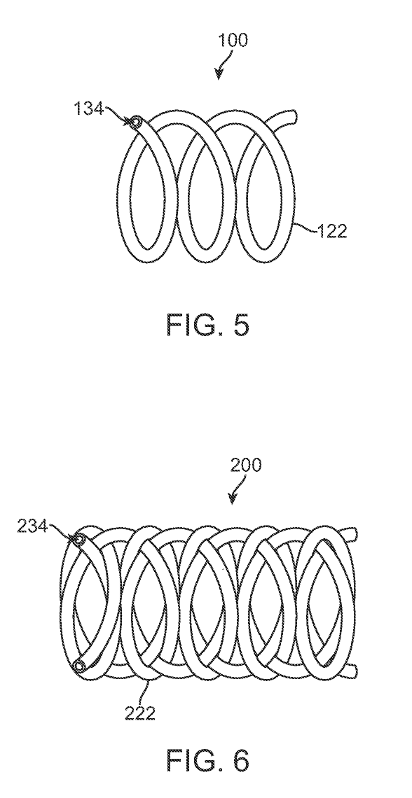

FIG. 4, illustrates another embodiment of an implantable device 10b made in accordance with the present invention. In this embodiment, hypotubes 22c are braided or woven into a mesh stent 10b in accordance with methods known in the art. In this embodiment, stent 10b comprises a plurality of hypotubes 22c braided in two opposing directions (clockwise and counter-clockwise) to form stent 10b. Hypotubes 22c comprise lumen 34b that is in fluid communication with one or more pores 42d to provide localized drug delivery at a treatment site. In one embodiment, pores 42d may be covered or plugged as described above.

In another embodiment, the hypotubes do not have drug release pores. In this embodiment, the drug is delivered by diffusion or a release of drug during degradation of a biodegradable hypotube. FIG. 5 illustrates one embodiment of a biodegradable implantable device 100 composed of at least one biodegradable hypotube 122. Aspects of implantable device 100 similar to or the same as those described above for the devices illustrated in FIGS. 1-4 will not be described further.

Biodegradable hypotube 122 is manufactured from materials that can biodegrade or bioerode over a period of time as a result of its exposure to blood and/or bodily fluid flow. In one embodiment, the material for use in a particular biodegradable implantable device 100 is chosen based on degradation properties such as, for example, length of time to degrade. The use of such biodegradable materials is beneficial in applications where subsequent removal of an implantable device from the patient's body is desired.

Biocompatible, biodegradable materials suitable for manufacturing biodegradable hypotubes 122 in accordance with the present invention can include, for example, biodegradable metals, metal alloys, polymers and combinations thereof. In one embodiment, the biodegradable metal is magnesium or a magnesium alloy. In another embodiment the biodegradable polymer includes, but is not limited to, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen, hyaluronic acid, poly-N-alkylacrylamides, poly depsi-peptide carbonate, polyethylene-oxide based polyesters, and combinations thereof.

Implantable device 100 further includes at least one drug and/or drug/carrier combination loaded into lumen 134 of hypotube 122. Drugs and carriers suitable for loading into implantable device 100 may be the same as or similar to those listed above in relation to FIGS. 1 to 4. Drugs that are suitable for release from the hypotubes of implantable device 100 include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like. In one embodiment, the drugs released include, but are not limited to, macrolide antibiotics including FKBP-12 binding agents. Exemplary drugs of this class include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid and zotarolimus (ABT-578). Additionally, other rapamycin hydroxyesters may be used in combination with the polymers of the present invention.

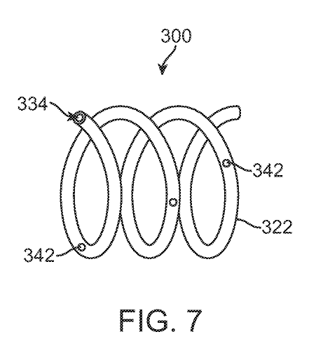

FIG. 6, illustrates another embodiment of a biodegradable implantable device 200 made in accordance with the present invention. In this embodiment, biodegradable hypotubes 222 are braided or woven into a stent 200 in accordance with methods known in the art. Biodegradable hypotubes 222 are composed of the same or similar materials as described in relation to FIG. 5. In this embodiment, stent 200 comprises a plurality of hypotubes 222 braided in two opposing directions (clockwise and counter-clockwise) to form stent 200. Hypotubes 222 comprise lumen 234. At least one drug or drug/carrier combination is loaded into lumen 234. The drugs and carriers suitable for implantable device 200 are the same as those described above. The at least one drug or drug/carrier combination is released after implantation upon the degradation of the biodegradable hypotubes 222 comprising implantable device 200.

FIG. 7 illustrates another embodiment of a biodegradable implantable device 300 in accordance with the present invention. Implantable device 300 comprises biodegradable hypotube 322 and a plurality of pores 342. As described above, pores 342 are in fluid communication with lumen 334. Lumen 334 is loaded with at least one drug or at least one drug/carrier combination as described above. In one embodiment, pores 342 of implantable device 300 are covered or plugged with a biodegradable material. In one embodiment, hypotube 322 is manufactured from a first biocompatible material that degrades at a first rate and the plurality of pores is plugged with a second biocompatible material that degrades at a second rate. In one embodiment, the second biocompatible material degrades at a rate that is higher than the degradation rate of the first biocompatible material. In one embodiment, the drug is substantially released from the pores prior to the degradation of the implantable device. In another embodiment, a plurality of biodegradable hypotubes 322 having pores 342 may be braided or woven to form implantable devices the same as or similar to implantable device 200 illustrated in FIG. 6.

In other embodiments, the biodegradable implantable devices illustrated in FIGS. 5 to 7 may be configured with compartments similar to those described above and illustrated in FIGS. 2a and 2b. In other embodiments having compartmentalized lumens, pores in fluid communication with the various compartments may be plugged with biodegradable material that degrades at various rates. In these embodiments, a stent may be manufactured that releases different drugs contained in separate compartments at different times throughout the degradation process of the biodegradable stent. In one embodiment, a biodegradable stent comprises a lumen having two compartments, each compartment containing a different drug. The compartments are in fluid communication with a plurality of pores that are plugged with biodegradable material. In this embodiment, the pores of the first compartment are plugged with a first biodegradable material that degrades at a rate different than a second biodegradable material used to plug pores of a second compartment. Those with skill in the art will appreciate that a stent may have any number of compartments and may be composed of many different biodegradable materials to suit a particular application. In one embodiment, a biodegradable stent is compartmentalized such that the lumen is divided substantially in half longitudinally. In this embodiment, pores disposed within a stent wall located on an outer surface of the hypotube release a first drug into or adjacent a vessel wall and pores disposed within a stent wall located on an inner, luminal surface release a second drug into the channel created by the stent upon delivery at the treatment site.

In another embodiment of the present invention a biodegradable implantable device is composed at least partially of at least one hypotube having multiple lumens. In one embodiment, the hypotube comprises at least two lumen arranged concentrically about a longitudinal axis. In this embodiment, each lumen may contain the same or different drug or therapeutic agent. In one embodiment, an inner lumen contains a first drug and a second lumen positioned radially outward of the first lumen contains a second drug. In this embodiment, the second drug elutes from the implantable device prior to the first drug.

In another embodiment of a multi-lumen hypotube, the hypotube comprises a compartmentalized hypotube where the compartments are arranged longitudinally along the length of the hypotube. The compartments may contain different drugs with different drug release profiles. In yet another embodiment of a multi-lumen hypotube, the hypotube includes two lumens running longitudinally along the length of the hypotube. In one embodiment, a first longitudinal compartment includes a first drug with a first drug release profile and the second longitudinal compartment includes a second drug with a second drug release profile.

Groupings of alternative elements or embodiments according to the invention disclosed herein are not to be construed as limitations. Each group member may be referred to individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

While several embodiments have described the implantable device as a stent, other medical devices would be advantageously formed from the hypotubes according to the teachings of the present invention. Exemplary implantable medical devices include, but are not limited to, stents, stent grafts, urological devices, spinal and orthopedic fixation devices, gastrointestinal implants, neurological implants, cancer drug delivery systems, dental implants, and otolaryngology devices.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

What is claimed is:

1. A biodegradable implantable device for delivering a drug to a treatment site comprising:
a biodegradable hypotube, the hypotube defining a lumen; and
at least one drug disposed within the lumen of the hypotube,
wherein the at least one drug is released from the lumen of the biodegradable hypotube.

2. The device of claim 1 wherein the implantable device comprises a stent.

3. The device of claim 2 wherein the stent comprises a plurality of hypotubes, wherein the plurality of hypotubes are in a configuration selected from the group consisting of a helical configuration, a braided configuration, a mesh configuration and a woven configuration.

4. The device of claim 1 wherein the biodegradable material comprising the hypotube comprises a material selected from the group consisting of biodegradable metals, biodegradable metal alloys, biodegradable polymers and combinations thereof.

5. The device of claim 4 wherein the biodegradable polymer is selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, and combinations thereof.

6. The device of claim 1 wherein the lumen includes at least two compartments.

7. The device of claim 6 wherein each of the compartments contains different drugs.

8. The device of claim 7 wherein each of the compartments exhibits different drug release profiles.

9. The device of claim 1 wherein the biodegradable hypotube includes a plurality of pores disposed within a wall of the hypotube, the plurality of pores in fluid communication with the lumen.

10. The device of claim 9 wherein the plurality of pores are plugged with a biodegradable material.

11. The device of claim 10 wherein the biodegradable material plugging the plurality of pores comprises a biodegradable material different than the biodegradable material comprising the hypotube.

12. The device of claim 9 wherein the plurality of pores are spaced along the hypotube to create different drug release profiles at different portions of the implantable device.

13. The device of claim 7 wherein the biodegradable hypotube includes a first plurality of pores disposed within a wall of the hypotube, the first plurality of pores in fluid communication with a first compartment of the lumen and a second plurality of pores disposed within the wall of the hypotube, the second plurality of pores in fluid communication with a second compartment of the lumen.

14. The device of claim 13 wherein a first drug within the first compartment has a first release profile and a second drug in the second compartment has a second release profile.

15. The device of claim 10 wherein the implantable device defines a channel and a majority of the plurality of pores are disposed on a portion of the hypotube in fluid communication with the channel.

16. The device of claim 10 wherein the implantable device defines a channel and a majority of the plurality of pores are disposed on the portion of the hypotube that is in fluid communication with a vessel wall.

17. The device of claim 1 wherein the at least one drug is combined with a biocompatible carrier before the drug is disposed within the lumen of the hypotube.

18. The device of claim 17 wherein the biocompatible carrier comprises a biodegradable material selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, mineral oils, caster oils, ethylene glycol, BHT and combinations thereof.

19. The device of claim 1 wherein the at least one drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

20. The device of claim 1 wherein the at least one drug is selected from the group consisting of sirolimus (rapamycin), tacrolimus (FK506), everolimus (certican), temsirolimus (CCI-779) and zotarolimus (ABT-578).
